(19) United States
(12) Patent Application Publication
(10) Pub. No.: US 2023/0233550 A1
(43) Pub. Date: Jul. 27, 2023

(54) TREATMENT FOR THE INFLAMMATORY BOWEL DISEASE

(71) Applicants: Mukul JAIN, Ahmedabad, Gujarat (IN); Amit JOHARAPURKAR, Ahmedabad, Gujarat (IN); Vishal PATEL, Ahmedabad, Gujarat (IN)

(72) Inventors: Mukul JAIN, Ahmedabad, Gujarat (IN); Amit JOHARAPURKAR, Ahmedabad, Gujarat (IN); Vishal PATEL, Ahmedabad, Gujarat (IN)

(21) Appl. No.: 17/928,719

(22) PCT Filed: May 31, 2021
(86) PCT No.: PCT/IB2021/054757

§ 371 Date: Nov. 30, 2022

(30) Foreign Application Priority Data
Jun. 1, 2020 (IN) 202021022965

(51) Int. Cl. A61K 31/4704; A61K 45/06; A61P 37/06; A61P 1/00

(52) U.S. Cl. CPC A61K 31/4704; A61K 45/06; A61P 1/00; A61P 37/06

(57) ABSTRACT

Present invention relates to development of compound of formula (Ia) for treatment of inflammatory bowel diseases. Invention also relates to pharmaceutical composition comprising therapeutically effective compound of formula (Ia) for treatment of inflammatory bowel diseases.

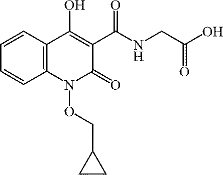 [SMILES: O=C(O)CNC(=O)c1c(O)c2ccccc2n(OCC2CC2)c1=O]

### FIELD OF THE INVENTION

The present invention relates to the development of therapeutic composition for the treatment of inflammatory bowel disease. In particular, the present invention provides a prolyl hydroxylase inhibitors or its pharmaceutically acceptable salt or suitable composition useful for the prevention or the treatment of inflammatory bowel disease. Specifically, present invention relates to development of compound of formula (Ia) for the treatment of inflammatory bowel disease. Invention also relates to pharmaceutical composition comprising compound of formula (Ia) for the treatment of inflammatory bowel disease.

### BACKGROUND OF THE INVENTION

Inflammatory bowel disease (IBD) is the chronic inflammatory disease affecting digestive track and specifically intestine. The principal types of IBD are Crohn’s disease and ulcerative colitis. The most common symptoms of IBD are diarrhea, abdominal pain, rectal bleeding or stool in blood, weight loss or fatigue. The most prevalent complication of inflammatory bowel disease is anemia. The exact cause of IBD is not yet known, but appears as a result of disturbed immune system. It can also be a triggered events from bacterial or viral infection (Abraham BP et al., 2017). The initial inflammation stated in gut mucosa which further, invades and affects the deep tissues. Mucosal inflammation alter metabolic activity and vascular tissue damage leading to tissue hypoxia which dampen healing processes (K Ko J et al., 2014). The therapeutic approach to treat IBD involves multidisciplinary management, including broad spectrum immuno-suppressants to reduce mucosal inflammation. 5-aminosalicylate-based compounds such as mesalamine used as a first line approach followed by corticosteroids. Azathioprine, 6-mercaptopurine and methotrexate and other immunomodulators have also been used as maintenance treatment (Fichna J, 2016). More recently the use of antibodies against TNF-alpha (anti-TNF) to reduce disease severity and progression have also being used (Colombel JF et al., 2017). Early use of antibodies, typically combined with immunomodulators may also increases risk of toxicity, infections and also increases risk of hepatosplenic T-cell lymphoma. The clinically used therapies are associated with several limitations. These limitations are: nonresponsiveness, high dose or long term treatment associated risk, high cost, increased risk of infection and malignancy with the use of biologic agents (Cohen BL et al., 2012). Indeed, the numbers of patients ultimately also requires surgical intervention, suggesting a significant need for new approaches to the treatment of IBD, which may be used in addition to or in combination with existing approaches (Cosnes J et al., 2012).

Oxygen is a regulator of acute and chronic inflammation. Hypoxia-inducible factors (HIFs: HIF-1 and HIF-2) senses oxygen levels in the tissues. HIF is widely expressed in inflammatory cells such as neutrophils, lymphocytes and other TH17 cells (Dang et al., 2011; McNamee et al., 2013; Shi et al., 2011; Walmsley et al., 2005). Intestinal inflammation demonstrate high degree of hypoxia, reflecting vascular disruption with increased oxygen demand by highly metabolically active infiltrating immune and inflamed resident cells (Colgan SP et al., 2010; Taylor CT et al., 2007). It may be possible that increased oxygen demands by infiltrating neutrophils generate reactive oxygen species and further increases oxygen consumption. HIF is crucial for providing barrier protection in inflamed epithelia in IBD (Karhausen J et al., 2004). It has been reported that DMOG a stabilizer of HIF protects against animal model of colitis (Cummins EP et al., 2008). Nuclear factor-κB (NF-κB) a master regulator of innate immune and inflammatory gene expression is principal among the HIF-independent transcription factors (Scholz et al., 2013). NF-kB signaling in intestinal epithelial cells is protective against the development of colitis (Greten FR et al., 2004). NF-kB -dependent genes include cytokines, adhesion molecules and regulators of cellular apoptosis (Luo JL et al., 2005). Prolyl hydroxylase (PHD) enzymes cause hydroxylation of HIF and thus directs HIF towards degradation. Inhibition of PHD can stabilize HIF thus increasing the availability of HIF at the site of inflammation. Several studies showed that pharmacologic PHD inhibition is protective in murine models of colitis (Cummins EP et al., 2008; Robinson A et al., 2008). Using a PHD inhibitor HIF can be stabilized and thus inflammation in IBD can be reduced and healing process can be increases. Compound of formula (Ia) is one of the PHD inhibitor currently in clinical trials. It is reported that treatment of compound of formula (Ia) stabilizes HIF and thus induces erythropoiesis in animal model of anemia (Jain et al., 2019; Joharapurkar et al., 2018). Thus, treatment of compound of formula (Ia), either systemically or locally, can be used in the treatment of IBD and other diseases of gut inflammation or impaired healing.

Some of the prolyl hydroxylase inhibitors and their uses have been disclosed in EP661269, WO2004108681, WO2007150011, WO2007070359, WO2008002576, WO2008067871, WO2008076425, WO2011007856, WO2012106472, WO2013043621, WO2013134660 and CN 110664814. CN 110664814 discloses pharmaceutical composition of Roxadustat for treating inflammatory bowel disease. US 8962530 discloses method of treating inflammatory bowel disease by using HIF prolyl hydroxylase is EGLN1, EGLN2, or EGLN3.

WO2014102818 also discloses propyl hydroxylase inhibitors of formula (I) and are useful for the treating anemia:

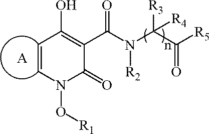

One of the Compound of formula (Ia) or its pharmaceutically acceptable salts disclosed in WO2014102818 has been found useful for prevention or treatment of inflammatory bowel disease.

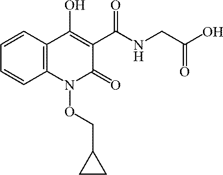 [SMILES: O=C(O)CNC(=O)c1c(O)c2ccccc2n(OCC2CC2)c1=O]

### OBJECTIVES OF THE INVENTION

In an embodiment, the present invention provides prolyl hydroxylase inhibitors suitable for the treatment of inflammatory bowel disease.

In another embodiment the present invention provides a suitable composition comprising prolyl hydroxylase inhibitors and suitable pharmaceutically acceptable excipients for the treatment of inflammatory bowel disease.

In an embodiment, the present invention provides a compound of formula (Ia) and their pharmaceutically acceptable salts suitable for the treatment of inflammatory bowel disease.

In another embodiment, the present invention provides a suitable pharmaceutical composition comprising
(a) the compound of formula (Ia) or its pharmaceutically acceptable salt
(b) Suitable pharmaceutical acceptable excipients; for the treatment of inflammatory bowel disease.

In another embodiment, the present invention provides the administration of compound of formula (Ia) and their pharmaceutically acceptable salts alone or in combination with other suitable agents as therapeutic agent for the treatment of inflammatory bowel disease.

In another embodiment, the present invention provides a method of treatment of inflammatory bowel disease using pharmaceutical composition of compound of formula (Ia).

### SUMMARY OF THE INVENTION

Present invention provides prolyl hydroxylase inhibitors for the prevention or treatment of inflammatory bowel disease. Specifically, invention is related to the propyl hydroxylase inhibitor compound of formula (Ia) for the treatment of inflammatory bowel disease. In addition, invention also provides a pharmaceutical composition comprising compound of formula (Ia) or its pharmaceutically acceptable salts useful in treatment of inflammatory bowel disease.

### BRIEF DESCRIPTION OF FIGURES

FIG. 1: The effect of compound of formula (Ia) on change in body weight and intestinal permeability in TNBS-induced colitis.

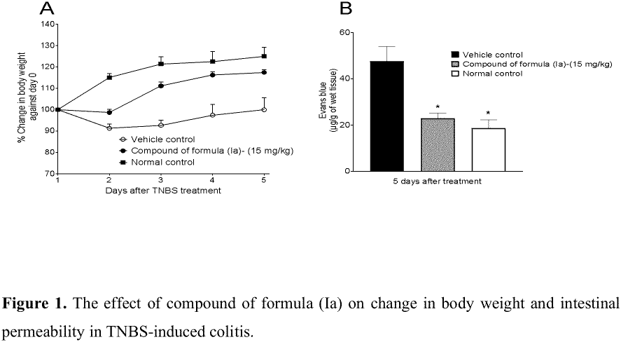

FIG. 2: The effect of compound of formula (Ia) on change in body weight, disease activity score and colon MPO in DSS-induced colitis.

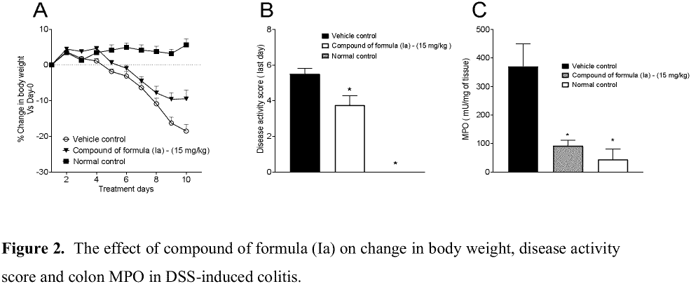

### DETAILED DESCRIPTION OF INVENTION

### Definitions

Term ‘treatment’ or ‘treating’ used herein anywhere in the specification means preventing, delaying or reducing the symptoms of the disease or disorder developing in the subject.

Term ‘subject’ used herein anywhere in specification means mammals, that include a human, an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

Term ‘pharmaceutically acceptable’ used herein anywhere in the specification means it is acceptable to use for both humans and animals.

Term ‘inflammatory bowel disease’ or ‘IBD’ is used for a disorder of digestive system of body that involves moderate of chronic inflammation of digestive tract.

Present invention provides prolyl hydroxylase inhibitors for the prevention or treatment of inflammatory bowel disease.

In one of the embodiment, prolyl hydroxylase inhibitors may selected from the Daprodustat, Desidustat, Molidustat and Vadadustat or pharmaceutically acceptable salts thereof.

In a preferred embodiment, prolyl hydroxylase inhibitor in present invention is Desidustat (Compound of formula (Ia)).

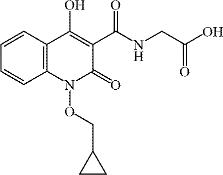 [SMILES: O=C(O)CNC(=O)c1c(O)c2ccccc2n(OCC2CC2)c1=O]

In an embodiment, compound of formula (Ia) or its pharmaceutically acceptable salts are used for treatment of inflammatory bowel disease.

Pharmaceutically acceptable salt of compound of formula (Ia) is either an organic salt or inorganic salt.

Organic salt can is selected from various amines such as methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethylbenzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3- Diaminopropane, (S)-α-naphthylethylamine, (S)-3-methoxyphenylethylamine, (S)-4- methoxyphenylethylamine, (S)-4-chlorophenylethylamine, (S)-4- methylphenylethylamine, cinchonine, cinchonidine, (-)-quinine, triethanolamine, imidazole, ethylenediamine, epolamine, morpholine 4-(2-hydroxyethyl), N-Ndiethylethanolamine, deanol, hydrabamine, betaine, adamantanamine, Ladamantanmethylamine, tritylamine, glucamine, N-methyl pyrrolidine, urea, procaine, hexane-1,6-diamine, 2-(2-aminoethoxy)ethanamine, N-methylmorpholine, and N-ethylmorpholine and the like.

Inorganic salt is in form of metal salts, selected from calcium, sodium, potassium, lithium, barium, strontium, magnesium, cesium, copper, cobalt, iron, manganese, lead, aluminum, cadmium, silver, zinc, ammonium and the like.

In another embodiment, the present invention provides a suitable pharmaceutical composition comprising;
(a) the compound of formula (Ia) or its pharmaceutically acceptable salt,
(b) Suitable pharmaceutical acceptable excipients;
and such pharmaceutical compositions are suitable for the treatment of inflammatory bowel disease.

In an embodiment there is provided a therapeutically effective amount of compound of formula (Ia) or its pharmaceutically acceptable salt is selected from 25 mg to 250 mg preferably 50 mg to 150 mg.

The pharmaceutical composition comprising compound of formula (Ia) or its pharmaceutically acceptable salts is administered to the subject by means of oral, parenteral or topical administration. In one of the preferred embodiment there is provided a pharmaceutical composition comprising compound of formula (Ia) or its pharmaceutically acceptable salt is administered by means of oral or parenteral administration.

In an embodiment, the present invention provides a pharmaceutical composition comprising compound of formula (Ia) or its pharmaceutically acceptable salts and optionally other pharmaceutically acceptable excipients.

The pharmaceutical acceptable excipients for solid or liquid oral pharmaceutical composition may be selected from binders, disintegrants, lubricants, fillers, glidants, wetting agents, solvents, buffers, surfactants, co-solvents, suspending agents, preservatives and the like.

Binders include, but are not limited to hypromellose 3 Cps, carbomers selected from carbopol, gellan, gum Arabic, hydrogenated vegetable oil, polymethacrylates selected from Eudragit, xanthan, lactose and combinations thereof and other such materials known to those of ordinary skill in the art.

Disintegrating agents include, maize starch, sodium starch glycolate, croscarmellose sodium, crospovidone, microcrystalline cellulose, modified com starch, sodium carboxymethyl starch, povidone, pregelatinized starch, agar, carboxymethyl cellulose calcium or sodium, colloidal silicon dioxide, chitosan, docusate sodium, hydroxyl propyl cellulose, magnesium aluminium silicate, maltose, methyl cellulose, polacrilin potassium, and alginic acid or suitable combinations thereof and other such materials known to those of ordinary skill in the art.

Lubricanting agents include, but are not limited to, magnesium stearate, stearic acid, silica, fats, zinc or sucrose or sodium or calcium stearate, castor oil, hydrogenated castor oil, . Polyethylene glycol and its derivatives, sodium stearyl fumarate, talc, or fatty acids including lauric acid, oleic acid, glyceryl behenate, glyceryl monostearate, and C1-C10 fatty acid or suitable combinations thereof and other such materials known to those of ordinary skill in the art.

Fillers include but not limited to, calcium carbonate, calcium phosphate, calcium sulphate, carboxymethylcellulose calcium, carboxymethylcellulose sodium, compressible sugar, confectioner’s sugar, dextrates, dextrin, dextrose, dibasic calcium phosphate dihydrate, dibasic calcium phosphate, fructose, glyceryl palmitostearate, glycine, hydrogenated vegetable oil-type 1, kaolin, lactose, maize starch, magnesium carbonate, magnesium oxide, maltodextrin, mannitol, microcrystalline cellulose, polymethacrylates, potassium chloride, powdered cellulose, pregelatinised starch, sodium chloride, sorbitol, starch, sucrose, sugar spheres, talc, tribasic calcium phosphate, xylitol.

Glidants include, but are not limited to, colloidal silica, calcium silicate, magnesium silicate, silicon hydrogel, cornstarch, talc, combinations thereof and other such materials known to those of ordinary skill in the art.

Wetting agents include, by way of example and without limitation, poloxamers, gelatin, casein, Glycerol mono-oleate, lecithin (phosphatides), gum acacia, cholesterol, tragacanth, stearic acid, benzalkonium chloride, calcium stearate, glycerol monostearate, cetostearyl alcohol, sodium lauryl sulphate, sodium dodecyl sulfate, salts of bile acids (taurocholate, glycocholate, cholate, deoxycholate, etc.), cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN), polyethylene glycols, polyoxyethylene stearates colloidal silicon dioxide, phosphates, sodium dodecylsulfate, carboxymethylcellulose calcium, carboxy methylcellulose sodium, methyl cellulose,hydroxyethylcellulose, hydroxylpropylcellulose, hydroxy propyl methyl cellulose phthalate, noncrystalline cellulose, magnesium aluminum silicate, triethanolamine, polyvinyl alcohol, and poly vinyl pyrrolidone (PVP) & their suitable combinations and other such materials known to those of ordinary skill in the art.

Diluents include, but are not limited to starch and its processed and co-processed derivertives, saccharides, di saccharides, sucrose, lactose, polysaccharides, cellulose, cellulose ethers, cellulose acetate, hydroxypropyl cellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di-basic calcium phosphate dihydrate, mannitol lactose anyhydrous, magnesium oxide, maltodextrin, maltose, pullulan, sodium alginate, sodium bicarbonate, calcium silicate, calcium sulphate, cell and tribasic calcium phosphate or suitable combinations thereof and other such materials known to those of ordinary skill in the art.

For parenteral formulations, pharmaceutically acceptable excipients may include but not limited to solvents, vehicles, buffers, preservatives, diluents, etc.

Solvents include, but not limited to polyethylene glycol-300, propylene glycol, isopropyl alcohol, pyrrolidone, ethanol and other aliphatic alcohols known to those of ordinary skill in art.

Preservative include, but not limited to pentetic acid, Potassium sorbate, Propionic acid, Propyl paraban, Sodium benzoate, Sodium borate, Sodium metabisulphite, Sodium propionate, Sodium sulphite, Benzyl alcohol, Sorbic acid, etc. and known to those of ordinary skill in art.

The pharmaceutical composition may be in the form of tablets, capsules, emulsion, syrup, granules, powder, suppositories, for oral administration or it may be in the form of reconstitution powder, suspensions, or sterile parenteral solutions for parenteral administration.

The pharmaceutical composition can be prepared by the conventional methods disclosed in standard textbooks and known to the person skilled in art.

In another embodiment, the present invention provides the administration of compound of formula (Ia) and their pharmaceutically acceptable salts alone or in combination with suitable secondary/additional therapeutic agent for the treatment of inflammatory bowel disease.

Suitable secondary/additional therapeutic agents for the combination may be selected from amino-salicylates, antibiotics, anti-inflammatory drugs, anti-diarrhoeal drugs, NSAIDs, JAK inhibitors, corticosteroids, immune modifying agents, and biologic agent (anti-tumor necrosis factor (TNF) agents).

In one of the embodiment, the dosing amount of the compound of formula (Ia) or its pharmaceutically acceptable salts in pharmaceutical composition may be in the range of 0.1 mg to 500 mg.

In another embodiment, the present invention provides a method of treatment of inflammatory bowel disease using compound of formula (Ia) or its pharmaceutically acceptable salts.

In yet another embodiment, the present invention also useful for method of treatment of disease associated with inflammatory bowel disease such as crohn’s disease, ulcerative colitis and other immune-mediated inflammatory diseases such as rheumatoid arthritis, systemic lupus erythematosus, etc.

In one of the embodiment, the pharmaceutical composition of other prolyl hydroxylase inhibitors such as Daprodustat,, Molidustat and Vadadustat or pharmaceutically acceptable salts thereof may be prepared using pharmaceutical excipients and methods as described above or known to the person skilled in the art including those processes disclosed in the prior art mentioned anywhere in the specification. The compound of formula (Ia) is prepared by the processes disclosed in WO2014102818 and other methods for preparation is disclosed in US 20190359574.

### EXAMPLES

### Example 1

Efficacy of compound of formula (Ia) in the treatment of inflammatory bowel disease is evaluated a by two methods:
i) TNBS-induced colitis
ii) DSS-induced colitis
Study protocol of both methods and results obtained from the evaluation are provided below

### Study Design and Treatment

### I) TNBS-Induced Colitis

Female BalbC mice (6-8 weeks old) were used in the study. Mice were sensitized by topical application of 1% trinitrobenzene sulfonic acid (TNBS) in acetone/olive oil solution (4:1). After 7 days of sensitization, mice were challenged with 2.5% TNBS solution by intra-rectal route administered 5 mL/kg of body weight under anesthesia. Mice were monitored for development of disease over 5 days. TNBS-treated mice were randomly divided into 2 groups: TNBS-control (TNBS) and TNBS + compound of formula (Ia) (15 mg/kg, orally). Treatment was started on one day before TNBS challenge (2.5 %) and continued up to day 5 after TNBS challenge (2.5%). Animals were monitored for body weight change daily for 5 days. Intestinal permeability was estimated by Evans blue method (Han ED et al., 2002).

### Ii) DSS-Induced Colitis

The 8 to 10 week old male C57 mice (18-20 g) were used in the study. Animals were divided into two experimental groups: vehicle control and compound of formula (Ia). Same day animals were provided 2% dextran sulfate sodium (DSS) solution in water for 7 days. Animals were treated with vehicle or compound of formula (Ia) (15 mg/kg) by oral route daily for 10 days. Body weight was recorded daily for 10 days. Disease activity score was evaluated based on stool consistency and rectal bleeding (Kim JJ et al., 2012). Relative colon length was measured. Colon myeloperoxidase (MPO) activity was estimated (Kim JJ et al., 2012).

### Results

### I) Effect of Compound of Formula (Ia) Treatment in TNBS-Induced Colitis

On 5^{th} days of TNBS- challenges in Balb/c mice, vehicle control group showed 99.9 ± 5.6% change in body weight while normal control treatment group showed 125.0 ± 4.3% change in body weight against day 1. By the treatment of compound of formula (Ia) weight was gain by 17.4 ± 1.4% when compared with vehicle control. TNBS- treatment increased intestinal permeability by 155.0 ± 33.8% when compared with normal control. Compound of formula (Ia) treatment decreased intestinal permeability by 51.7 ± 4.4% when compared against vehicle control.

### Ii) Effect of Compound of Formula (Ia) Treatment in DSS-Induced Colitis

After ten days of treatment, vehicle controlled mice showed 18.5 ± 1.9% weight loss when compared to day 1. Normal control animals gained body weight by 5.6 ± 1.7% when compared with day 1. Compound of formula (Ia) treatment increased body weight by 49.0 ± 12.9% against vehicle control. Vehicle control group showed increase in disease activity score of 5.5 ± 0.3 when compared with no score of normal control animals. Compound of formula (Ia) treatment decreased score by 31.8 ± 9.8% when compared with vehicle control. Colon myeloperoxidase activity was increased in vehicle control animals from 44.2 ± 36.1 to 370 ± 79.3 mU/mg of colon. Compound of formula (Ia) treatment reduced colon MPO by 74.9 ± 5.0% against vehicle control.

### REFERENCES

1. Abraham BP, Ahmed T, Ali T. Inflammatory bowel disease: pathophysiology and current therapeutic approaches. In Gastrointestinal Pharmacology 2017 (pp. 115-146). Springer, Cham.

2. K Ko J, K Auyeung K. Inflammatory bowel disease: etiology, pathogenesis and current therapy. Current pharmaceutical design. 2014, 20(7):1082-96.

3. Fichna J. Inflammatory Bowel Disease Treatment. Pharmacol Rep. 2016, 68 (4), 787-788.

4. Colombel JF, Mahadevan U. Inflammatory bowel disease 2017: innovations and changing paradigms. Gastroenterology. 2017, 152(2): 309-12.

5. Cosnes J, Nion-Larmurier I, Beaugerie L, et al. Impact of the increasing use of immunosuppressants in Crohn’s disease on the need for intestinal surgery. Gut 2005;54:237-241

6. Cohen BL, Torres J, Colombel JF. Immunosuppression in inflammatory bowel disease: how much is too much? Curr Opin Gastroenterol 2012; 28:341-348

7. Colgan SP, Taylor CT. Hypoxia: an alarm signal during intestinal inflammation. Nat Rev Gastroenterol Hepatol 2010, 7:281-287.

8. Taylor CT, Colgan SP. Hypoxia and gastrointestinal disease. J Mol Med (Berl) 2007, 85:1295-1300.

9. Greten FR, Eckmann L, Greten TF, et al. IKKbeta links inflammation and tumorigenesis in a mouse model of colitis-associated cancer. Cell 2004, 18:285-296.

10. Karhausen J., Furuta G. T., Tomaszewski J. E., Johnson R. S., Colgan S. P. and Haase V. H. Epithelial hypoxia-inducible factor-1 is protective in murine experimental colitis. J. Clin. Invest. 2004, 114, 1098-1106.

11. Cummins E. P., Seeballuck F., Keely S. J., Mangan N. E., Callanan J. J., Fallon P. G. and Taylor C. T. The hydroxylase inhibitor dimethyloxalylglycine is protective in a murine model of colitis. Gastroenterology 2008, 134, 156-165.

12. Luo JL, Kamata H, Karin M. The anti-death machinery in IKK/NF-kappaB signaling. J Clin Immunol 2005, 25:541-550.

13. Robinson A, Keely S, Karhausen J, et al. Mucosal protection by hypoxiainducible factor prolyl hydroxylase inhibition. Gastroenterology 2008, 134:145-155.

14. Dang, E. V., Barbi, J., Yang, H.Y., Jinasena, D., Yu, H., Zheng, Y., Bordman, Z., Fu, J., Kim, Y., Yen, H.R., Luo, W., Zeller, K., Shimoda, L., Topalian, S.L., Semenza, G.L., Dang, C. V., Pardoll, D.M., Pan, F., Control of TH17/Treg balance by hypoxia-inducible factor 1. Cell 2011, 146, 772-784.

15. McNamee, E.N., Korns Johnson, D., Homann, D., Clambey, E.T. Hypoxia and hypoxia-inducible factors as regulators of T cell development, differentiation, and function. Immunol. Res. 2013, 55, 58-70

16. Shi, L.Z., Wang, R., Huang, G., Vogel, P., Neale, G., Green, D.R., Chi, H. HIF1α-dependent glycolytic pathway orchestrates a metabolic checkpoint for the differentiation of TH17 and Treg cells. J. Exp. Med. 2011, 208, 1367-1376.

17. Walmsley, S.R., Print, C., Farahi, N., Peyssonnaux, C., Johnson, R.S., Cramer, T., Sobolewski, A., Condliffe, A.M., Cowburn, A.S., Johnson, N., Chilvers, E.R.Hypoxia-induced neutrophil survival is mediated by HIF-1α-dependent NF-κB activity. J. Exp. Med. 2005, 201, 105-115.

18. Jain, M., Joharapurkar, A., Patel, V., Kshirsagar, S., Sutariya, B., Patel, M., Patel, H., Patel, P.R. Pharmacological inhibition of prolyl hydroxylase protects against inflammation-induced anemia via efficient erythropoiesis and hepcidin downregulation. Eur. J. Pharmacol. 2019, 843, 113-120.

19. Joharapurkar, A.A., Pandya, V.B., Patel, V.J., Desai, R.C., Jain, M.R.Prolyl Hydroxylase Inhibitors: A Breakthrough in the Therapy of Anemia Associated with Chronic Diseases. J. Med. Chem. 2018, 61, 6964-6982

20. Kim JJ, Shajib MS, Manocha MM, Khan WI. Investigating intestinal inflammation in DSS-induced model of IBD. JoVE (Journal of Visualized Experiments). 2012, 1(60):e3678.

21. Han ED, MacFarlane RC, Mulligan AN, Scafidi J, Davis AE. Increased vascular permeability in C1 inhibitor-deficient mice mediated by the bradykinin type 2 receptor. The Journal of clinical investigation. 2002, 109(8):1057-63.

1. Pharmaceutical composition comprising
(a) the compound of formula (Ia) or its pharmaceutically acceptable salt 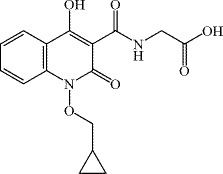 [SMILES: O=C(O)CNC(=O)c1c(O)c2ccccc2n(OCC2CC2)c1=O]
(b) suitable disintegrating agents, suitable lubricating agents and optionally other suitable pharmaceutical acceptable excipients for the treatment of inflammatory bowel disease.

2. Pharmaceutical composition as claimed in claim 1 wherein pharmaceutically acceptable salts are selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethylbenzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (-)-quinine, triethanolamine, imidazole, ethylenediamine, epolamine, morpholine 4-(2-hydroxyethyl), N-Ndiethylethanolamine, deanol, hydrabamine, betaine, adamantanamine, Ladamantanmethylamine, tritylamine, glucamine, N-methyl pyrrolidine, urea, procaine, hexane-1,6-diamine, 2-(2-aminoethoxy)ethanamine, N-methylmorpholine, and N-ethylmorpholine; calcium, sodium, potassium, lithium, barium, strontium, magnesium, cesium, copper, cobalt, iron, manganese, lead, aluminum, cadmium, silver, zinc, ammonium and the like.

3. Pharmaceutical composition as claimed in claim 1 for the treatment of inflammatory bowel disease that may include chron’s disease, ulcerative colitis and other immune mediated inflammatory diseases such as rheumatoid arthritis and systemic lupus erythematosus.

4. Pharmaceutical composition as claimed in claim 1 wherein therapeutically effective amount of compound of formula (Ia) is selected from range of 25 mg to 250 mg.

5. Pharmaceutical composition as claimed in claim 1 wherein pharmaceutical acceptable excipients are selected from binders, disintegrants, lubricants, fillers, glidants, wetting agents, solvents, buffers, surfactants, co-solvents, suspending agents and preservatives.

6. Pharmaceutical composition as claimed in claim 5 wherein disintegrating agents are selected from maize starch, sodium starch glycolate, croscarmellose sodium, crospovidone, microcrystalline cellulose, modified com starch, sodium carboxymethyl starch, povidone, pregelatinized starch, agar, carboxymethyl cellulose calcium or sodium, colloidal silicon dioxide, chitosan, docusate sodium, hydroxyl propyl cellulose, magnesium aluminium silicate, maltose, methyl cellulose, polacrilin potassium, and alginic acid or suitable combinations thereof.

7. Pharmaceutical composition as claimed in 5 wherein lubricating agents are selected from lubricants include magnesium stearate, stearic acid, silica, fats, zinc or sucrose or sodium or calcium stearate, castor oil, hydrogenated castor oil, . Polyethylene glycol and its derivatives, sodium stearyl fumarate, talc, or fatty acids including lauric acid, oleic acid, glyceryl behenate, glyceryl monostearate, and C₁-C₁₀ fatty acid or suitable combinations thereof.

8. Pharmaceutical composition as claimed in claim 5 wherein diluents are selected from maltitol, lactitol, microcrystalline cellulose, magnesium or calcium or sodium carbonate, lactose, lactose monohydrate, di-calcium phosphate, compressible sugars, di-basic calcium phosphate dihydrate, mannitol lactose anyhydrous, magnesium oxide, maltodextrin, maltose, pullulan, sodium alginate, sodium bicarbonate, calcium silicate, calcium sulphate, cell and tribasic calcium phosphate or suitable combinations thereof.

9. Pharmaceutical composition as claimed in claim 5 wherein binders are selected from hypromellose 3 Cps, carbomers selected from carbopol, gellan, gum Arabic, hydrogenated vegetable oil, polymethacrylates selected from Eudragit, xanthan, lactose and suitable combinations thereof.

10. Pharmaceutical composition as claimed in claim 5 wherein fillers are selected from calcium carbonate, calcium phosphate, calcium sulphate, carboxymethylcellulose calcium, carboxymethylcellulose sodium, compressible sugar, confectioner’s sugar, dextrates, dextrin, dextrose, dibasic calcium phosphate dihydrate, dibasic calcium phosphate, fructose, glyceryl palmitostearate, glycine, hydrogenated vegetable oil-type 1, kaolin, lactose, maize starch, magnesium carbonate, magnesium oxide, maltodextrin, mannitol, microcrystalline cellulose, polymethacrylates, potassium chloride, powdered cellulose, pregelatinised starch, sodium chloride, sorbitol, starch, sucrose, sugar spheres, talc, tribasic calcium phosphate, xylitol and suitable combinations thereof.

11. Pharmaceutical composition as claimed in claim 5 wherein glidants are selected from colloidal silica, calcium silicate, magnesium silicate, silicon hydrogel, corn-starch, talc, and suitable combinations thereof.

12. Pharmaceutical composition as claimed in claim 5 wherein wetting agents are selected from poloxamers, gelatin, casein, Glycerol mono-oleate, lecithin, gum acacia, cholesterol, tragacanth, stearic acid, benzalkonium chloride, calcium stearate, glycerol monostearate, cetostearyl alcohol, sodium lauryl sulphate, sodium dodecyl sulfate, salts of bile acids, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates colloidal silicon dioxide, phosphates, sodium dodecylsulfate, carboxymethylcellulose calcium, carboxy methylcellulose sodium, methyl cellulose, hydroxyethylcellulose, hydroxylpropylcellulose, hydroxy propyl methyl cellulose phthalate, noncrystalline cellulose, magnesium aluminum silicate, triethanolamine, polyvinyl alcohol, and poly vinyl pyrrolidone (PVP) and suitable combinations thereof.

13. Pharmaceutical composition as claimed in claim 1 wherein pharmaceutical composition is to be administered by means of oral or parenteral administration.

14. Pharmaceutical composition as claimed in claim 1 wherein pharmaceutical composition is in the form of tablets, capsules, emulsion, syrup, granules, powder, suppositories, suspensions, reconstitution powder or sterile solutions.

15. Pharmaceutical composition as claimed in claim 1 further comprising additional therapeutic agents are selected from classes - amino-salicylates, antibiotics, anti-inflammatory drugs, anti-diarrhoeal drugs, NSAIDs, JAK inhibitors, corticosteroids, immune modifying agents, and biologic agent (anti-tumor necrosis factor (TNF) agents).

16. Compound of formula (Ia) or its pharmaceutically acceptable salts for the treatment of inflammatory bowel diseases wherein compound of formula (Ia) is 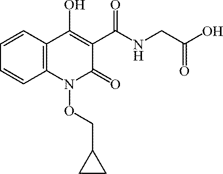 [SMILES: O=C(O)CNC(=O)c1c(O)c2ccccc2n(OCC2CC2)c1=O]
.

17. Compound as claimed in claim 16 is for the treatment of inflammatory bowel disease that may include chron’s disease, ulcerative colitis and other immune mediated inflammatory diseases such as rheumatoid arthritis and systemic lupus erythematosus.

18. Method of treating inflammatory bowel diseases comprising administering a therapeutically effective amount of a compound of formula (Ia) or its pharmaceutically acceptable salts to a patient wherein compound of formula (Ia) is 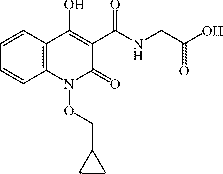 [SMILES: O=C(O)CNC(=O)c1c(O)c2ccccc2n(OCC2CC2)c1=O]
.
